(19) United States
(12) Patent Application Publication
(10) Pub. No.: US 2025/0242100 A1
(43) Pub. Date: Jul. 31, 2025

(54) AUTOMATED CATHETER AND CHEST TUBE DEVICES AND RELATED SYSTEMS

(71) Applicant: Tessefi Innovations, Inc., Rocky Mount, NC (US)

(72) Inventor: Mengesha Keelen, Rocky Mount, NC (US)

(21) Appl. No.: 18/705,036

(22) PCT Filed: Dec. 7, 2022
(86) PCT No.: PCT/US2022/052077

§ 371 Date: Apr. 26, 2024

Related U.S. Application Data
(60) Provisional application No. 63/286,752, filed on Dec. 7, 2021.

(51) Int. Cl. A61M 1/00; A61M 1/04; A61M 27/00

(52) U.S. Cl. CPC A61M 1/70; A61M 1/04; A61M 27/00; A61M 2202/0496; A61M 2205/12; A61M 2205/128; A61M 2205/14; A61M 2205/3306; A61M 2205/331; A61M 2205/3334; A61M 2205/3389

(57) ABSTRACT

A smart fluid discharge monitoring device can have a housing; a frame within the housing; a cartridge positioned within an upper shell of the housing, the cartridge having a window, through which a discharge fluid can flow; a door removably attached to the housing, the door delimiting an interior region of the housing below the upper shell; a tube connected to the cartridge; a plurality of tanks removably secured within the interior region, the tanks receiving the discharge fluid from the cartridge. The smart fluid discharge monitoring device can detect one or more optical characteristics of the discharge fluid through the window as the discharge fluid flows through the cartridge. The smart fluid discharge monitoring device can be a smart catheter or a smart chest tube.

### CROSS-REFERENCE TO RELATED APPLICATIONS

This application claims priority to U.S. Provisional Patent Application No. 63/286,752, which was filed on Dec. 7, 2021, the entire contents of which is incorporated herein by reference.

### TECHNICAL FIELD

The subject matter herein generally relates to the field of catheters, chest tubes, and related devices. The subject matter herein more particularly relates to, automated catheter devices and related systems, including urinary catheters and chest tubes.

### BACKGROUND

Modern medical catheters, particularly Foley catheters, are used to collect urine in patients while admitted to hospitals and medical facilities. However, the amount of urine collected by the catheter is not automatically calculated, and the data is not available electronically via a computer. Instead, urine output must be manually measured or observed, and the data manually charted or recorded when time permits. This can be detrimental to the patient because if a nurse, Nursing Assistant, technician or other medical professional forgets to chart the amount of periodic output a provider will assume there was no urine output. There are numerous potentials for errors including, but not limited to spilled urine and incorrect or imprecise measurements. This is crucial in providing proper medical care. For example, if a patient is admitted with a diagnosis such as diabetes insipidus, proper recording and utilization of urine output can be a life or death proposition.

Similarly, chest tubes are used to collect air, fluid, pleural effusion, blood, chyle, or pus from the intrathoracic space of a patient while admitted to hospitals and medical facilities, and/or otherwise under medical care. However, the amount of air, fluid, pleural effusion, blood, chyle, or pus collected by the chest tube is not automatically calculated, and the data is not available electronically via a computer. Instead, the amount of air or fluid output must be manually measured or observed, and the data manually charted or recorded when time permits. Similar to the urine catheter issues described above, if the nurse, or other medical professional, fails to chart the periodic air, fluid, pleural effusion, blood, chyle, or pus collected from the patient, a provider will assume there was no output. These types of charting are critical for receiving the proper treatment and, just like with the catheter, the potential consequence for error could be deadly. For example, if a patient is admitted with a particular condition, proper recording and utilization of air, fluid, pleural effusion, blood, chyle, or pus output can be a life or death proposition.

Thus, what is needed is an automated system delivering results in real-time or a device that collects, measures and/or records urine output and/or air, fluid, pleural effusion, blood, chyle, and/or puss output with minimal or no effort by medical personnel. Such need is addressed by the devices and system described herein.

### SUMMARY

According to an example embodiment, a smart fluid discharge monitoring device is provided herein, the smart fluid discharge monitoring device comprising a housing; a frame within the housing; a cartridge positioned within an upper shell of the housing, wherein the cartridge comprises a window, through which a discharge fluid is configured to flow; a door removably attached to the housing, the door delimiting an interior region of the housing below the upper shell; a tube connected to the cartridge; and a plurality of tanks removably secured within the interior region, wherein the tanks are configured to receive the discharge fluid from the cartridge; wherein the smart fluid discharge monitoring device is configured to detect one or more optical characteristics of the discharge fluid through the window as the discharge fluid flows through the cartridge.

In some embodiments, the smart fluid discharge monitoring device comprises a controller; the cartridge comprises a valve assembly for each of the plurality of tanks; and the controller is configured to control actuation of the valve assemblies between an open position and a closed position.

In some embodiments, the smart fluid discharge monitoring device comprises a tank occupancy sensor configured to detect when a tank is installed within the smart fluid discharge monitoring device.

In some embodiments of the smart fluid discharge monitoring device, each tank is attached to the frame via a corresponding tank mount; each tank mount is connected to the frame by a load cell; and the load cell is configured to output a signal that corresponds to a volume of the discharge fluid within the tank attached to the corresponding tank mount

In some embodiments of the smart fluid discharge monitoring device, the controller receives the signal from the load cell and, when the controller determines that one of the tanks is full, or within a predefined percentage of full, the controller is configured to close the valve assembly for the full tank and to open the valve assembly of another of the tanks.

In some embodiments of the smart fluid discharge monitoring device, each tank mount is connected to the frame only by the load cell, in a cantilevered manner.

In some embodiments of the smart fluid discharge monitoring device, each valve assembly of the cartridge comprises an actuator configured to engage with and move a valve body of a same valve assembly between an open position, in which the valve body is positioned to allow the discharge fluid to flow into a corresponding one of the plurality of tanks from the cartridge, and a closed position, in which the valve body is positioned to block the discharge fluid from flowing into the corresponding one of the plurality of tanks from the cartridge.

In some embodiments of the smart fluid discharge monitoring device, for each valve assembly of the cartridge, the valve body comprises a sloped surface against which the actuator is configured to engage in a sliding or rolling manner as the valve body moves between the open and closed positions.

In some embodiments of the smart fluid discharge monitoring device, each valve assembly comprises an arm that is attached to the actuator; for each valve assembly of the cartridge, the valve body comprises, at an end of the sloped surface that corresponds to the open position for the valve body when engaged by the actuator, a recess; the arm comprises, at a distal end thereof, a roller; and when the roller is engaged within the recess, the valve body is held in the open position until the actuator is energized to retract the arm, which disengages the roller from the recess.

In some embodiments of the smart fluid discharge monitoring device, each valve assembly is configured to default to the valve body being in the closed position unless the roller is engaged within the recess.

In some embodiments of the smart fluid discharge monitoring device, the smart fluid discharge monitoring device is configured to measure a flow rate of the discharge fluid through the cartridge.

In some embodiments, the smart fluid discharge monitoring device comprises a backlight configured to illuminate the discharge fluid through the window and a camera configured to detect the one or more optical characteristics of the discharge fluid through the window.

In some embodiments of the smart fluid discharge monitoring device, the one or more optical characteristics comprise turbidity and/or total dissolved solids.

In some embodiments of the smart fluid discharge monitoring device, the cartridge is positioned within the upper shell, which is configured to block exterior light from entering into the upper shell.

In any of the embodiments of the smart fluid discharge monitoring device, the smart fluid discharge monitoring device is a smart fluid discharge monitoring device, configured for connection to a fluid discharge monitoring device configured for evacuation of the discharge fluid from a patient to whom the fluid discharge monitoring device is attached.

In any of the embodiments of the smart fluid discharge monitoring device, the smart fluid discharge monitoring device is a smart chest tube, configured for connection to a chest tube configured for evacuation of the discharge fluid from a patient to whom the chest tube is attached.

According to another example embodiment, a method of monitoring a discharge fluid from a patient, the method comprising: providing a smart fluid discharge monitoring device, the smart comprising a housing; a frame within the housing; a cartridge positioned within an upper shell of the housing, wherein the cartridge comprises a window, through which a discharge fluid is configured to flow; a door removably attached to the housing, the door delimiting an interior region of the housing below the upper shell; a tube connected to the cartridge; and a plurality of tanks removably secured within the interior region; the method comprising connecting a discharge tube attached to a patient to the tube of the cartridge; detecting one or more optical characteristics of the discharge fluid through the window as the discharge fluid flows through the cartridge; and receiving the discharge fluid into one of the tanks from the cartridge.

In some embodiments of the method, the cartridge comprises a valve assembly for each of the plurality of tanks, the method comprising providing a controller that controls actuation of the valve assemblies between an open position and a closed position.

In some embodiments, the method comprises detecting, via a tank occupancy sensor, when a tank is installed within the smart fluid discharge monitoring device.

In some embodiments, the method comprises attaching each tank to the frame via a corresponding tank mount; connecting each tank mount to the frame by a load cell; and outputting, from the load cell, a signal that corresponds to a volume of the discharge fluid within the tank attached to the corresponding tank mount

In some embodiments, the method comprises receiving, at the controller, the signal from the load cell and, when the controller determines that one of the tanks is full, or within a predefined percentage of full, closing, using the controller, the valve assembly for the full tank and opening, using the controller, the valve assembly of another of the tanks.

In some embodiments of the method, each tank mount is connected to the frame only by the load cell, in a cantilevered manner.

In some embodiments of the method, each valve assembly of the cartridge comprises an actuator that engages with a valve body of a same valve assembly, the method comprising using the actuator to move the valve body between an open position, in which the valve body is positioned to allow the discharge fluid to flow into a corresponding one of the plurality of tanks from the cartridge, and a closed position, in which the valve body is positioned to block the discharge fluid from flowing into the corresponding one of the plurality of tanks from the cartridge.

In some embodiments of the method, for each valve assembly of the cartridge, the valve body comprises a sloped surface against which the actuator engages in a sliding or rolling manner as the valve body moves between the open and closed positions.

In some embodiments of the method, each valve assembly comprises an arm that is attached to the actuator; for each valve assembly of the cartridge, the valve body comprises, at an end of the sloped surface that corresponds to the open position for the valve body when engaged by the actuator, a recess; the arm comprises, at a distal end thereof, a roller; and when the roller is engaged within the recess, the valve body is held in the open position until the actuator is energized to retract the arm, which disengages the roller from the recess.

In some embodiments of the method, each valve assembly defaults to the valve body being in the closed position unless the roller is engaged within the recess.

In some embodiments, the method comprises measuring a flow rate of the discharge fluid through the cartridge.

In some embodiments, the method comprises illuminating, using a backlight, the discharge fluid through the window; and detecting, using a camera, the one or more optical characteristics of the discharge fluid through the window.

In some embodiments of the method, the one or more optical characteristics comprise turbidity and/or total dissolved solids.

In some embodiments of the method, the cartridge is positioned within the upper shell, which blocks exterior light from entering into the upper shell.

In any of the embodiments of the method, the discharge tube is a catheter and the smart fluid discharge monitoring device is a smart catheter.

In any embodiments of the method, the discharge tube is a chest tube and the smart fluid discharge monitoring device is a smart chest tube.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIG. 1 is a perspective view of a smart catheter device, according to an embodiment of the presently disclosed subject matter.

FIG. 2 is a perspective internal view of the smart catheter device of FIG. 1, according to an embodiment of the presently disclosed subject matter.

FIG. 3 is a partially exploded view of the smart catheter device of FIG. 1, according to an embodiment of the presently disclosed subject matter.

FIG. 4 is a perspective view of a valve assembly of the smart catheter device of FIG. 1, according to an embodiment of the presently disclosed subject matter.

FIGS. 5 and 6 are perspective views of a portion of the valve assembly of FIG. 4, in which the valve is shown actuated in respective closed and open positions, according to an embodiment of the presently disclosed subject matter.

FIG. 7 Is a perspective view of a tank mount and a frame of the smart catheter device of FIG. 1, according to an embodiment of the presently disclosed subject matter.

FIG. 8 is a perspective view of a tank for use in the smart catheter device of FIG. 1, according to an embodiment of the presently disclosed subject matter.

FIG. 9 is a perspective sectional view of the frame, the tank mount, and the tank of the smart catheter device of FIG. 1, according to an embodiment of the presently disclosed subject matter.

FIG. 10 is a side internal view of the smart catheter device of FIG. 1, according to an embodiment of the presently disclosed subject matter.

FIG. 11 is a perspective internal view of a fluid evaluation portion of the smart catheter device of FIG. 1, according to an embodiment of the presently disclosed subject matter.

FIG. 12 is a perspective internal view of a portion of the smart catheter device of FIG. 1, in which a presence sensor is shown being used to detect proper alignment and/or insertion of a tank within the smart catheter device, according to an embodiment of the presently disclosed subject matter.

FIG. 13 is a partially exploded view of the smart catheter device of FIG. 1, according to an embodiment of the presently disclosed subject matter.

FIG. 14 is a perspective view of a control board attached to the frame of the smart catheter device of FIG. 1, according to an embodiment of the presently disclosed subject matter.

FIG. 15 is a photograph of an example imaging device of the smart catheter device of FIG. 1, according to an embodiment of the presently disclosed subject matter.

FIG. 16 is an illustration of an example load cell of the smart catheter device of FIG. 1, according to an embodiment of the presently disclosed subject matter.

FIGS. 17A-D show various aspects regarding a tank level measuring subsystem of the smart catheter device of FIG. 1, according to an embodiment of the presently disclosed subject matter.

FIG. 18 is a schematic block diagram of components of the smart catheter device of FIG. 1, according to an embodiment of the presently disclosed subject matter.

FIG. 19 is a front view of an alternate example embodiment of a smart catheter device.

FIG. 20 is a top view of the smart catheter device of FIG. 19.

FIG. 21 is a side view of the smart catheter device of FIG. 19.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The present subject matter provides automated or smart catheter systems and devices and automated or smart chest tube systems and devices. The term “smart fluid discharge monitoring device” can be used interchangeably herein to refer to the example embodiments of both the smart catheters and the smart chest tubes disclosed herein. In one aspect, the present subject matter provides smart catheter systems and devices for draining, storing, and measuring urine from a patient and warning or alerting healthcare officials once the urine levels get to a certain level or the device malfunctions. In similar aspect, the present subject matter provides smart chest tube systems and device for draining, storing, and measuring bodily fluids from a patient and warning or alerting healthcare officials once the bodily fluid levels reach a certain threshold. While the following terms are believed to be well understood by one having ordinary skill in the art, the following definitions are set forth to facilitate explanation of the presently disclosed subject matter.

Unless defined otherwise, all technical and scientific terms used herein have the same meaning as commonly understood to one having ordinary skill in the art to which the presently disclosed subject matter belongs. Although, any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a vial” can include a plurality of such vials, and so forth.

Unless otherwise indicated, all numbers expressing quantities of length, diameter, width, and so forth used in the specification and claims are to be understood as being modified in all instances by the terms “about” or “approximately”. Accordingly, unless indicated to the contrary, the numerical parameters set forth in this specification and attached claims are approximations that can vary depending upon the desired properties sought to be obtained by the presently disclosed subject matter.

As used herein, the terms “about” and “approximately,” when referring to a value or to a length, width, diameter, temperature, time, volume, concentration, percentage, etc., is meant to encompass variations of in some embodiments±20%, in some embodiments±10%, in some embodiments ±5%, in some embodiments ±1%, in some embodiments±0.5%, and in some embodiments±0.1% from the specified amount, as such variations are appropriate for the disclosed apparatuses and devices.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “subject”, “individual”, and “patient” are used interchangeably herein, and refer to an animal, especially a mammal, for example a human, to whom treatment or monitoring, with a device or system as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears.

Urinary catheters are used to collect urine from patients who are admitted to hospitals and medical facilities, as well as at home and for chronic use. The urine output is manually collected and measured by nurses and other medical professionals. Unfortunately, there are no known automated systems and/or devices for performing this important task. Instead, urine output must be manually measured or observed, and the data manually charted or recorded, usually hours after the collection event, or after hours of collection of a volume of urine, so as to provide a volumetric flow rate that is averaged over this period of time. This is a crucial step in providing proper medical care, but due to the manual nature of this task it can easily be overlooked or delayed. Automated, or smart, catheter systems and/or devices that collect, measure, and/or record urine output from a patient with minimal or no effort by medical personnel would improve efficiency, accuracy, best practice, health care effectiveness and successful patient outcomes. The disclosed devices and systems fill this unmet need. Particularly, the disclosed devices and systems automatically collect, measure, calculate and record urine output and other related data. In some embodiments, such catheters and catheter systems can be referred to as a precise catheter, smart catheter, electronic catheter, and the like, or precise Foley, smart Foley, electronic Foley, and the like, all of which can be used interchangeably herein.

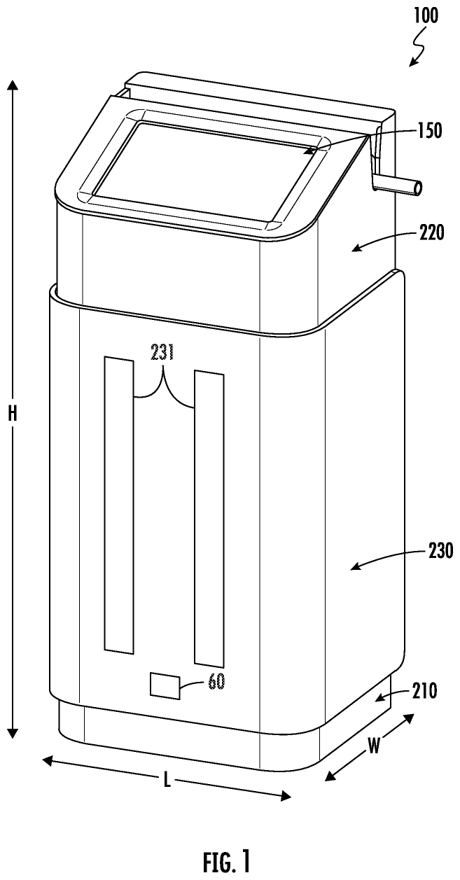

Referring to FIG. 1, a perspective view of an example embodiment of a smart catheter, generally designated 100, is shown therein. Such a smart catheter 100 is of the type that can be, for example, attached (e.g., removably attached) to a side of a hospital bed. In the example embodiment shown, the smart catheter 100 has a housing, or enclosure, that contains therein a subset of (e.g., a majority of) the components of the smart catheter 100. The housing of the smart catheter includes a base 210; a frame 200, which is attached to the base 210; an upper shell 220, which is attached to the frame 200, and a door 230 removably attached to the frame 200. The door 230 is positioned between the base 210 and the upper shell 220 such that, when the door is in a closed position, the components of the smart catheter 100 that are contained within and/or attached to the frame 200 are concealed by the door 230 and, when the door 230 is in an open position, the components of the smart catheter 100 that are contained within and/or attached to the frame are visible, accessible, and/or removable from the exterior of the smart catheter 100. In some embodiments, the door 230 is pivotably and/or rotatably connected, or attached, to the frame (e.g., such as may occur by connection therebetween by a hinge or other suitable structure), such that the door 230 is movable from the open position into the closed position and from the closed position into the open position, as well as into positions between the open position and the closed position. In some embodiments of the smart catheter 100, the door 230 is removable and/or detachable from the frame 200 (e.g., in the manner of disengaging a plurality of clips) for accessing the components contained within and/or attached to the frame 200. In some embodiments of the smart catheter 100, the door 230 includes a marking (e.g., a pattern, grooves, or other indicia) indicating the direction in which the door 230 opens (e.g., by pivoting about the attachment point) from the closed position. This marking advantageously allows medical professionals to easily determine which way to open the door to increase efficiencies when trying to inspect or exchange components contained within the structures (e.g., the frame 200, the base 210, the upper shell 220, and the door 230) that are generally regarded as forming the housing of the smart catheter 100. The door 230 advantageously has a height that extends entirely from at least the bottom edge or surface (e.g., in the direction H shown in FIG. 1) of the upper shell 220 to at least the top edge or surface (e.g., in the direction H shown in FIG. 1) of the base 210.

The smart catheter 100 comprises, in a position on and/or recessed within the upper shell 100 of the housing, a display 150. The display 150 is positioned on a surface (e.g., an upper, or upward-facing, surface) of the upper shell 220 so that the display 150 is visible and accessible from an exterior of the smart catheter 100. In the example embodiment shown, the display 150 is a touchscreen display (e.g., a display that is configured to receive user inputs via the user touching one or more locations on the display). In some embodiments, the display 150 can be a conventional display screen and the smart catheter 100 can have, provided thereon, one or more user inputs (e.g., buttons, a keyboard, switches, etc.) provided on an external surface thereof, such as, for example and without limitation, on the upper shell 220 and adjacent to (e.g., on the same surface as) the display 150. In some embodiments, the display 150 can be a conventional display screen and the smart catheter 100 may be configured to connect to an authenticated user device via a suitable wireless connection (e.g., WiFi®, Bluetooth®, and the like) and to receive inputs from the authenticated user device for, e.g., setting operational parameters on the smart catheter 100.

The smart catheter 100 also comprises a tube 10 that extends from a port in the exterior surface of the housing (e.g., from the side of the upper shell 220 in FIG. 1), the tube 10 being configured for connection to a catheter for collecting fluid from a patient to whom the catheter is connected. In some embodiments, the smart catheter 100 comprises a lighting element 60 that is configured to emit a light, so as to allow for illumination of the surroundings of the smart catheter 100 (e.g., the room in which it is positioned) in the manner of, for example, a night light, thereby allowing nurses, doctors, and other health professionals to view the display 150 and also the surrounding areas of the smart catheter 100 to avoid, for example, trip hazards, without disturbing the patient by turning on more disruptive lighting sources (e.g., overhead, or ceiling-mounted, lights) in the room.

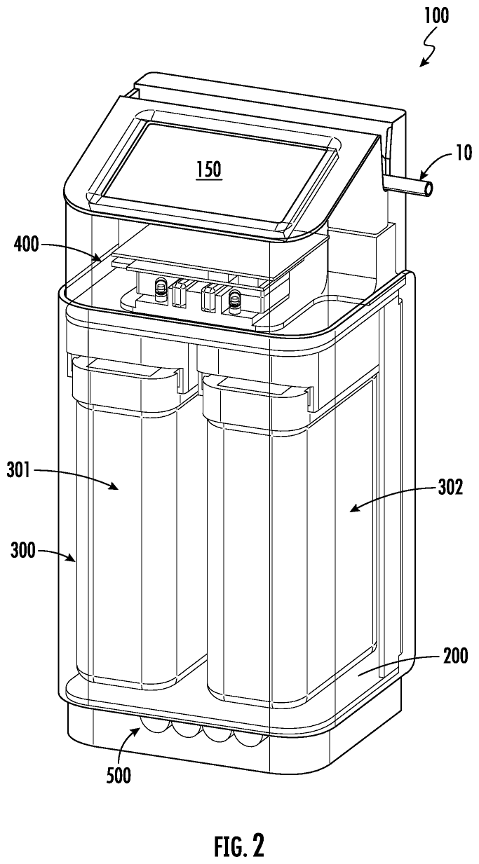

As shown in FIG. 2, the smart catheter 100 shown therein comprises, in the base 210 thereof, a battery, generally designated 500, for providing power to the smart catheter 100 for continuing operation of the smart catheter 100 when the smart catheter 100 is, for example, disconnected from an external power source (e.g., is unplugged from an electrical outlet). In some embodiments, the base 210 and the battery 500 contained therein is removable or detachable from the frame 200, in the manner of a removable battery pack, for replacement and/or charging of a depleted battery 500 with a base 210 containing a fully charged battery 500. In some embodiments, the smart catheter 100 can be, either in addition to the battery 500 or in lieu of the battery 500, powered by a wired power connection (e.g., in the form of a cable or cord) to an electrical outlet. Advantageously, the smart catheter 100 is configured to trigger and sound an alarm (e.g., an audible claxon and/or visual signal), as described herein, when the voltage and/or current from the power supply (e.g., the electrical outlet) to which the smart catheter 100 is connected is inadequate and/or when there is a malfunction detected for the power connector, the battery 500, or other power source to which the smart catheter 100 can be operably connected. Examples of events and/or operational states that can be the basis for the generation of an alarm state can include, for example and without limitation, the first tank 301 is full; the second tank 302 is full; the first and second tanks 301, 302 are full; one or both of the first and second tanks 301, 302 are almost full (e.g., 70% or more, 80% or more, 90% or more); the first tank 301 needs to be removed and replaced; the second tank 302 needs to be removed and replaced; battery 500 low power remaining state; battery 500 depleted power state; discharge fluid flow rate higher than prescribed threshold; and discharge fluid flow rate less than prescribed threshold.

In some embodiments, the smart catheter 100 is operable using speech recognition (e.g., comprises a microphone and is configured to use voice-recognition software thereon). In support of this voice-activated functionality, some embodiments of the smart catheter 100 presently disclosed herein comprise speakers and an audio input device, such as a microphone, which allow a user to use speech commands and/or natural language to issue commands, inquiries, and/or requests to the smart catheter 100. Moreover, in some such embodiments, the one or more processors (e.g., in the electronics 400) of the smart catheter 100 are configured to operate an artificial intelligence program, which is configured to receive spoken commands and/or inquiries and to respond with additional audio feedback and/or perform tasks associated with the commands and/or inquiries received. In this way, any of the parameters, actions, services, or performances that can be executed by the smart catheter 100, whether automatically or manually by touching or otherwise interacting with the smart catheter 100, can also be executed via voice command(s) using speech recognition in some such embodiments.

In FIG. 2, a perspective view of the example embodiment of the smart catheter 100 of FIG. 1 is shown, but with the upper shell 220, door 230, and base 210 illustrated as wireframe structures, such that the components of the smart catheter 100 within the housing are visible. The upper shell 220 of the housing forms therein an upper region in which electronics, generally designated 400, of the smart catheter 100 are operably positioned. A tank region, generally designated 300, is formed within a volumetric region defined by the door 230 and the frame 200. The smart catheter 100 contains, within the tank region 300, two or more tanks, a first tank 301 and a second tank 302, as well as the tank mounts 240 associated therewith. The tank mounts 240 are attached to the frame 200 and support one of the first and second tanks 301, 302 within the smart catheter.

In the example embodiments disclosed herein, the smart catheter 100 comprises a plurality of tanks and each of the first and second tanks 301, 302 is connected to and supported from and/or within the smart catheter 100 (e.g., via rigid attachment to the frame 200) by a corresponding tank mount 240. Thus, the quantity of tank mounts 240 that are provided for a smart catheter 100 corresponds to (e.g., is the same as) the quantity of tanks 301, 302 that the smart catheter 100 is configured to hold and/or utilize for allowing a flow of a fluid into a selected one of the tanks 301, 302 during use. While the example embodiment of the smart catheter 100 shown has a first tank 301 and a second tank 302, any suitable quantity of tanks 301, 302, i.e. a plurality of tanks, can be used in the smart catheter 100 to allow for redundant operation of the smart catheter 100 with any desired quantity for the plurality of tanks 301, 302.

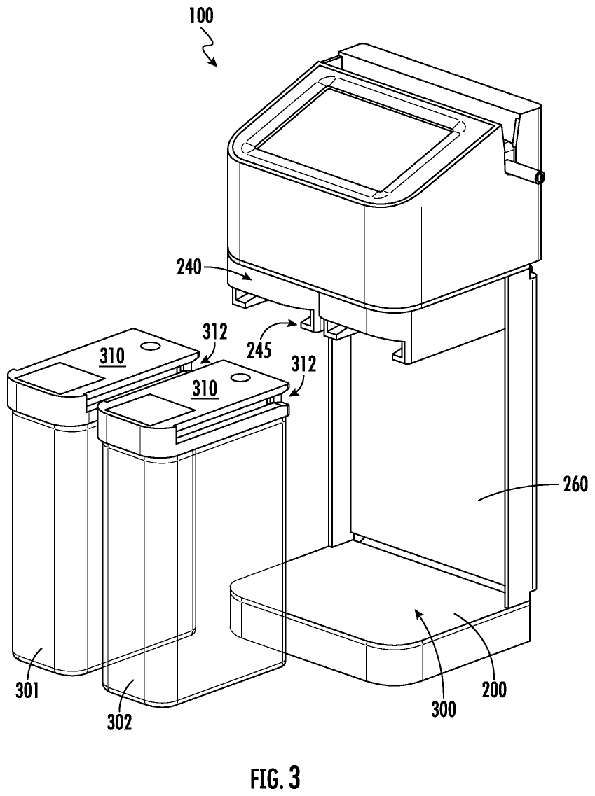

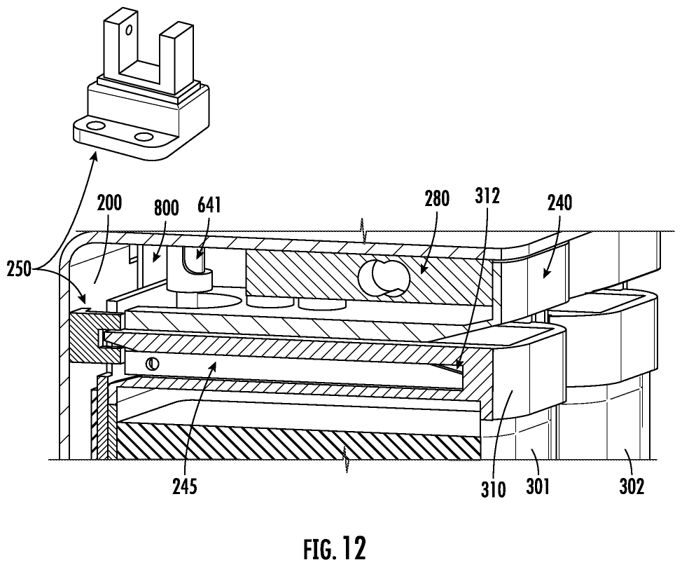

The tanks are suspended from a corresponding tank mount, such that, when properly installed in the smart catheter 100 and aligned within the tank mount 240, each of the tanks 301, 302 is not in direct contact with any other structure of the smart catheter 100 with the sole exception of it being connected to a corresponding one of the tank mounts 240. Stated differently, when installed in the smart catheter 100, each tank 301, 302 is only in direct contact with the corresponding tank mount 240. As shown in FIG. 3, the tanks 301, 302 are slidingly engageable with, and removable from, the tank mounts 240. In the example embodiments disclosed herein, the tanks 301, 302 are not keyed to any particular tank mount 240, so that each tank 301, 302 can be mounted to any of the tank mounts 240 interchangeably. To allow this sliding engagement functionality, each tank mount 240 can in some embodiments comprise a pair of opposing rails (or other suitable guiding structure), generally designated 245, that extend in a direction of insertion of one of the tanks 301, 302. Each tank 301, 302 comprises, formed in the tank lid 310 thereof, a corresponding pair of opposing slots, generally designated 312, the slots 312 being formed so as to extend in the same direction of insertion as the rails 245. An example illustration of the engagement of a rail 245 of the tank mount 240 within a slot 312 of the tank lid 310 is shown in FIG. 12.

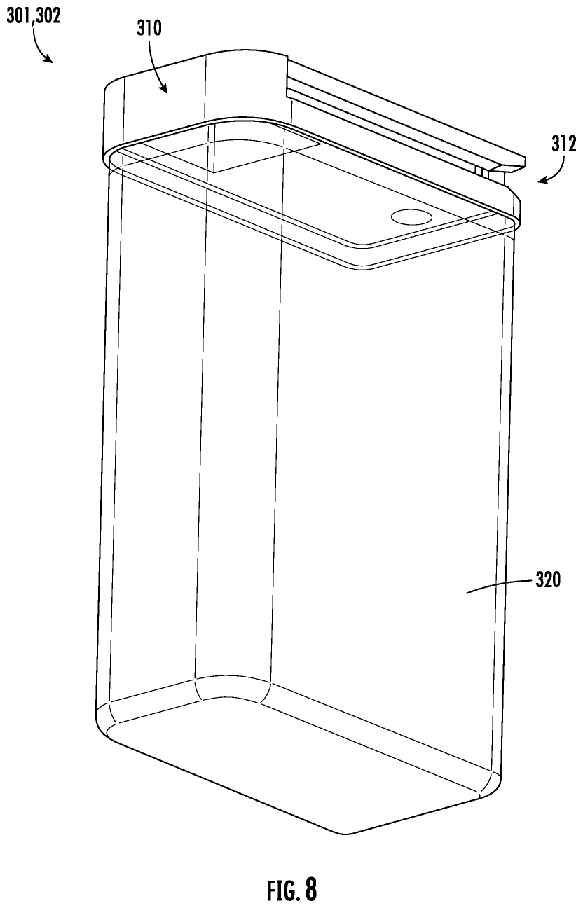

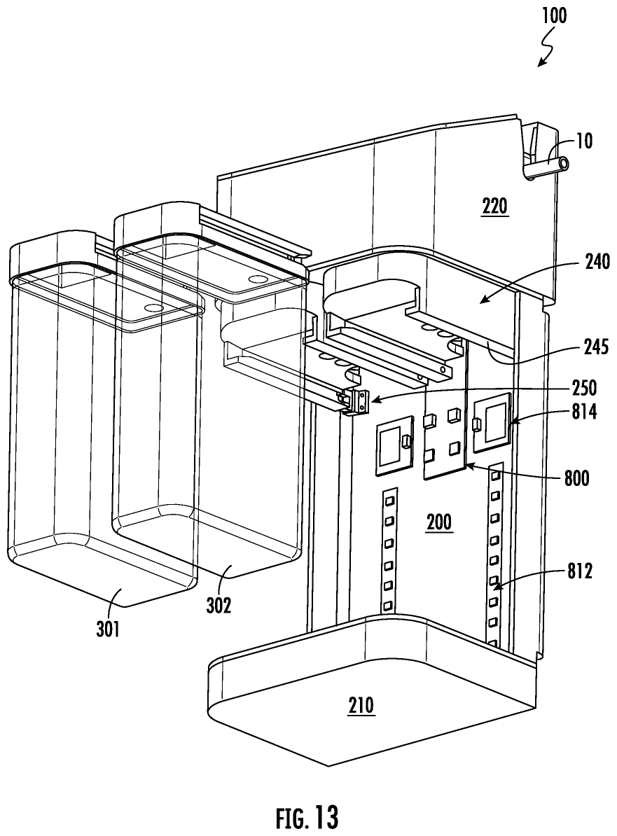

The tanks 301, 302 comprise a two-part construction, as shown in FIG. 8, in which a tank lid 310 is securely (e.g., removably) attached to a container 320. The container 320 is advantageously translucent (e.g., clear or substantially clear), so that a fluid level (e.g., as determined by height within the container 320) within the container 320 can readily be determined and/or observed visually by a user. As shown in FIG. 13, a plurality of backlights, generally designated 812, can be positioned on the frame 200, or another suitable structure of the smart catheter 100, in a position adjacent to the tanks 301, 302, such that the backlights 812 emit a light that can pass through, from back to front (e.g., from the direction of the vertical wall of the frame 200 towards the door 230), the tanks 301, 302 to increase visibility of a liquid within one or both tanks 301, 302. In some embodiments, the illumination (e.g., intensity of light emitted) of the backlights 812 is controlled, for example, by a control input variable that can be modified by accessing a menu via the display 150, by actuating a physical switch of the smart catheter 100 that is accessible by a user, and/or by a door switch that is activated when the door 230 is not in the closed position.

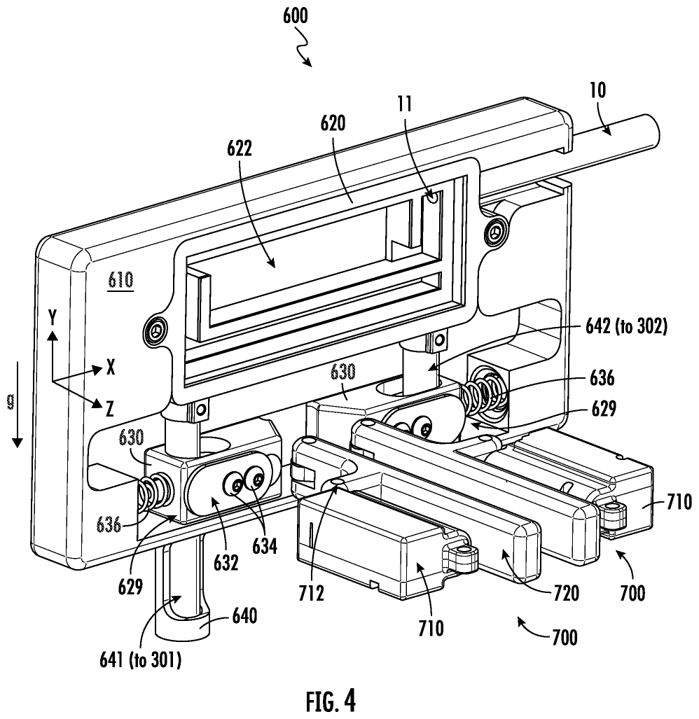

To control a flow of discharge fluid into a designated one of the plurality of tanks 301, 302, the smart catheter 100 comprises a cartridge, generally designated 600, and an actuator assembly, generally designated 700, example aspects of which are shown in FIGS. 4-6. The cartridge 600 comprises a main body 610, in which a recess is formed for receiving therein an inlet tube 10, a window housing 620, one or more (e.g., at least two, or a plurality of) drain tubes 641, 642, and a valve assembly, generally designated 629. The inlet tube 10 is connected to the window housing 620 via an inlet hole, generally designated 11, by which discharge fluid is introduced into the window housing 620. At least both lateral sides (e.g., in the X-direction) of the window housing 620 are at least translucent, so as to define a window, generally designated 622. In order to allow for accurate analysis of optical characteristics of the discharge fluid as it flows through the window housing 620 (e.g., from the inlet hole 11 and into one of the drain tubes 641, 642), it is particularly advantageous for the lateral sides of the window housing 620 to be transparent (e.g., clear or substantially clear). Drain tubes 641, 642 are attached to the window housing 620 at designated locations (e.g., at corresponding respective drain holes that are provided in and entirely through the bottom wall of the window housing 620 at a position that is directly above a corresponding one of the drain tubes 641, 642). Each drain tube 641, 642 is rigidly attached to the cartridge 600 by a guide 640 to ensure proper alignment thereof with an inlet formed in the tank lid 310 of a corresponding one of the tanks 301, 302, so as to ensure that the discharge fluid is consistently transferred from the window housing 620 into a designated one of the tanks 301, 302. It is advantageous for the flow of the discharge fluid through the window housing to be driven primarily by gravity, so as to provide increased reliability for the smart catheter 100. In order to allow the flow of the discharge fluid through the window housing 620 to be driven primarily by gravity, the inlet hole 11 is provided vertically above (e.g., with respect to the gravity vector, g) the discharge holes, to which the drain tubes 641, 642 are attached.

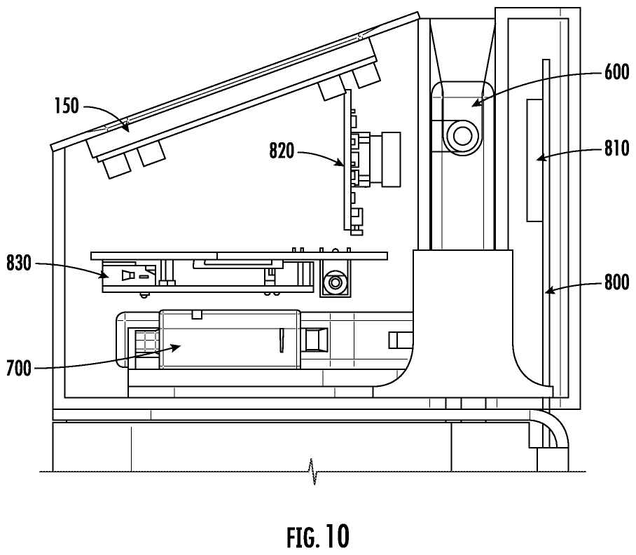

The actuator assembly 700 is used, in conjunction with the controller 830 (see, e.g., FIG. 10) and the valve assembly 629 contained in the cartridge 600, to selectively control into which of the tanks 301, 302 of the smart catheter 100 that the discharge fluid is dispensed. In the example embodiment shown herein, the valve assemblies 629 are in the form of pinch valves, which are biased into the closed position to prevent a flow of the discharge fluid from the cartridge 600 into one of the tanks 301, 302. For each drain tube 641, 642, a valve body 630 is provided. Each drain tube 641, 642 passes through a slot, generally designated 631, which is formed vertically through (e.g., in the axial direction of the drain tube, referred to hereinafter as the “y-direction”) the entirety of the corresponding valve body 630. A slider 650 is rigidly attached to the main body 610 of the cartridge 600, so as to prevent relative movement between the slider 650 and the main body 610 of the cartridge 600. The slot formed in each valve body 630 further extends so as to pass through (e.g., in the z-direction, which is perpendicular to the y-direction) the entirety of the corresponding valve body 630. The valve body 630 is inserted over the slider 650, such that the slider 650 is positioned within the portion of the slot 631 that extends in the y-direction, and a retention plate 632 is rigidly attached (e.g., by fasteners, such as rivets, screws, bolts, and the like) to the slider 650 to prevent movement of the valve body 630 in the z-direction relative to the main body 610 of the cartridge 600 during operation of the smart catheter. The valve body is thus free to move in the x-direction, which is perpendicular to the y- and z-directions.

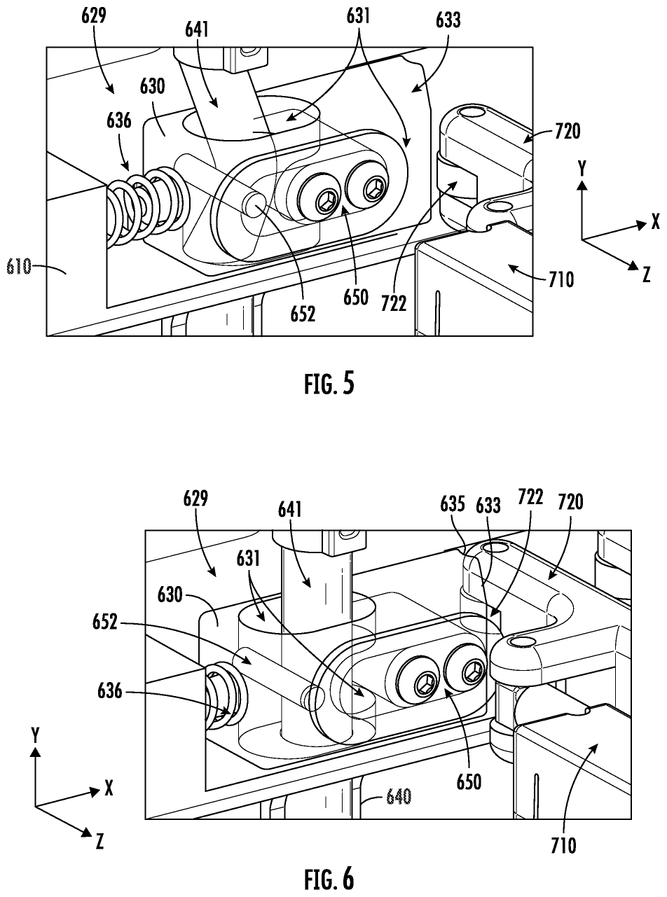

Each valve body 630 comprises, internal thereto, a fixed pin 652, or other suitably rigid protrusion, which extends within the valve body 630 in the z-direction. The pin 652 is positioned vertically within the volumetric region defined by the portion of the slot 631 that is formed vertically (e.g., in the z-direction) through the valve body 630. When the valve body 630 is in the closed position, shown in FIG. 5, the drain tube 641, 642 is squeezed between the pin 652 and the slider 650, such that no fluid can pass through the drain tube 641, 642 while the valve body 630 is in this closed position. Thus, the drain tube 641, 642 is made from a material that is readily deformable (e.g., in elastic deformation), such that the drain tube 641, 642 can be deformed to entirely close the cross-section thereof. An example of such a material particularly advantageous for forming the drain tubes 641, 642 is silicone tubing.

As shown in FIG. 5, the drain tube 641, 642 is deformed, when the valve body 630 is in the closed position, between the slider 650 and the pin 652, to the point that no fluid can pass through the drain tube 641, 642. A spring 636, or other suitable biasing member, is provided between the main body 610 of the cartridge 600 and the valve body 630, such that the spring 636 exerts a biasing force in the x-direction such that, absent actuation by the actuator assembly 700, the drain body 630 remains in the closed position to ensure that no passage of the discharge fluid through the drain pipe 641, 642 can occur unless the valve body 630 is moved into the open position, such as by the actuator assembly 700. This biasing of the valve body 630 in the closed position by the spring 636 is advantageous because it can prevent the undesired flow of the discharge fluid from the window housing 620 when, for example, one of the tanks 301, 302 has been removed from within the tank region 300 between the frame 200 and the door 230.

The smart catheter 100 comprises, associated with each valve body 630, an actuator assembly 700. The quantity of valve bodies 630 and of the actuator assemblies 700 is advantageously the same. Furthermore, there are advantageously the same quantity of each of the valve bodies 630, the actuator assemblies 700, and the tanks 301, 302. Thus, in the example embodiment disclosed herein, the smart catheter 100 comprises two (2) valve bodies 630, two (2) actuator assemblies 700, and two (2) tanks 301, 302. In some embodiments, the smart catheter 100 comprises an ambient light sensor that is used to determine the intensity of light in the immediate environment in which the smart catheter 100 is deployed.

As shown in FIGS. 4-6, each actuator assembly 700 comprises an actuator 710 that is attached to an arm 720. In the example embodiment shown, the actuator 710 is a linear actuator, which is connected to the arm 720 for driving the arm 720 in a substantially linear direction (e.g., in the z-direction). The valve body 630 has a contact surface 633, which is inclined relative to the x-and z-directions, in the manner of a sloping block. A roller 722 is pivotably affixed to the distal end of the arm 720 and is configured to engage in a rolling manner against the contact surface 633 of the valve body 630 as the arm 720 is extended by the actuator 710, such that the valve body 630 is moved from the closed position (shown in FIG. 5) into the open position (shown in FIG. 6). The use of the roller 722 for engagement of the arm 720 against the valve body 630 is advantageous for reducing friction between the arm 720 and the valve body 630, thereby providing for improved, reduced, actuation forces compared for, for example, a sliding frictional interface between the arm 720 and the valve body 630.

From the closed position in FIG. 5, the actuator 710 is configured to, upon receipt of an actuation signal, extends the arm 720 in the z-direction, towards the valve body 630. When the roller 722 contacts the contact surface 633 of the valve body 630, the valve body 630 begins to move (e.g., in a sliding manner, guided by the slider 650 within the horizontally-extending portion of the slot 631) in the x-direction, towards the open position shown in FIG. 6. As the actuator 710 continues to extend the arm 720 in the z-direction, the roller 722 rotates against the contact surface 633 of the valve body 630 and the valve body 630 continues to move in the x-direction, towards to the open position. The movement in the x-direction of the valve body 630 is proportional to a distance by which the arm 720 is extended by the actuator 710 over at least a portion of the length of travel, or extension, of the arm 720.

As shown in FIG. 6, the valve body 635 has a rest surface 635, against which the roller 722 is positioned when the arm 720 is in the fully extended position. The rest surface 635 can have a generally or substantially flat surface or can have a contour that is rounded, or concave, curved towards the roller 722 so that the roller 722 and the rest surface 635 can engage with each other and so that a retraction movement of the roller 722 is resisted by the curved shape of the rest surface 635. In some embodiments, the rest surface 635 has a substantially similar curvature to that of the roller 722. The engagement of the roller 722 with the rest surface 635 can be advantageous because, unlike if the roller 722 remained in contact with the inclined contact surface 633 of the valve body 630, in the example embodiment disclosed herein the valve body 630 only exerts the biasing force (e.g., from the spring 636 acting on the valve body 630) on the roller 722 in the x-direction. If the roller 722 were to remain in contact with the inclined contact surface 633 of the valve body 630 when the valve body 630 is in the open position, the biasing force of the spring 636 acting on the roller 722 through the valve body would have a vector, or Cartesian component, in the z-direction, in the direction of retraction of the arm 720, which may cause unintended movement of the valve body 630 into the closed position if the position of the valve body 630 were not monitored. Furthermore, by there being substantially no z-direction component of the biasing force on the arm, the actuator 710 can be de-energized (e.g., turned off, so as to not exert a force on the arm 720) at all times other than when the actuator 710 is moving the arm 720 into and between the open and closed positions. A position of the arm 720 can be determined by feedback provided to the controller 830 by the actuator 710.

The components of the two valve assemblies are arranged, relative to each other, in mirror image about a plane that bisects the cartridge 600 vertically (e.g., in the y- and z-directions), such that the cartridge 600 comprises two valve assemblies that are substantially identical to each other, other than being arranged as mirror images of each other. Thus, for example, while the valve body 630 of the first valve assembly (e.g., shown in FIG. 5) is configured to slide in the negative x-direction (e.g., left. as shown in the orientation of FIG. 4)) when moving from the closed position into the open position, the valve body 630 of the second valve assembly is configured to slide in the positive x-direction (e.g., right, as shown in the orientation of FIG. 4) when moving from the closed position into the open position. This opposite direction of movement of the respective valve bodies 630 of the first and second valve assemblies is thus achieved by the arrangement of the components of the first and second valve assemblies as mirror images of each other, even though the actuation direction (e.g., the direction of extension) of the arm 720, as driven by the actuator 710 is the same for both of the first and second valve assemblies.

The cartridge 600 is removable from the smart catheter 100 and the presence of the cartridge 600 within the smart catheter 100 is determined by a sensor that is configured to send a signal to the controller 830 based on whether or not the cartridge 600 is detected by the sensor. In some embodiments, the smart catheter 100 comprises a plurality of tabs, each tab being associated with one of the discharge tubes 641, 642, each tab being configured to extend over a corresponding discharge tube 641, 642 when fluid flow therethrough is disabled by the corresponding valve assembly 629, such that drop formation of the discharge fluid at the discharge end of such discharge tube 641, 642 is inhibited.

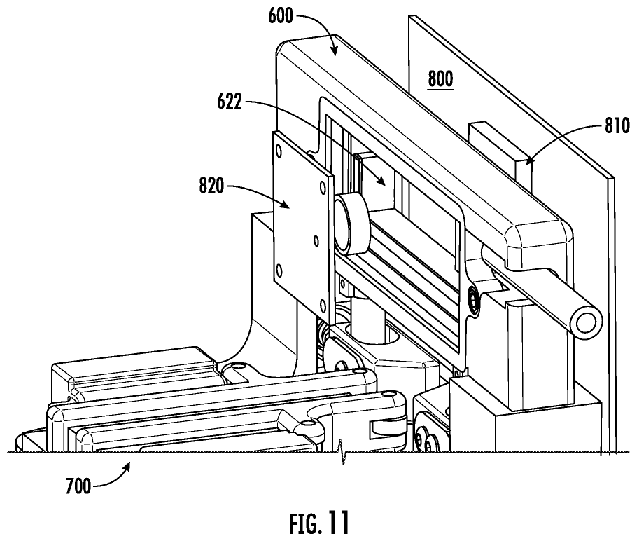

As noted elsewhere herein, the window 622 of the window housing 620 comprises a translucent material or, advantageously, transparent material. As shown in FIG. 11, a camera, generally designated 820, is positioned on a first side of the window 622, the camera 820 being oriented such that a field of view thereof is incident on, or pointing through, the window 622. A backlight 810 is positioned on a second side of the window 622, which is an opposite side from the camera 820. The backlight 810 is configured to emit light for illuminating the discharge fluid within the window housing 620 and the camera 820 is configured to detect, determine, and monitor optical characteristics of the discharge fluid that is illuminated by the backlight 810; examples of such optical characteristics can include opacity, color, and the like. Thus, the camera 820 is configured to send a video signal and/or a series of images of the discharge fluid through the window 622 to a controller (e.g., 830, see FIG. 10), which is configured to continuously monitor the optical characteristics of the discharge fluid within the window housing 620 and to trigger an alert when the optical characteristics of the discharge fluid are determined to have changed in any of a plurality of prescribed undesirable manners. The camera 820 is positioned within an optical zone that enables precise measurement of the optical characteristics of the discharge fluid (e.g., urine) within the window housing 620 and through the window 622. The camera 820 is configured at least as an RGB sensor to measure red, green, and blue color levels of the discharge fluid through the window 622. The upper shell 220 is constructed so as to be substantially entirely enclosed, thereby preventing light infiltration within the upper shell 220, which would alter the optical characteristics of the discharge fluid, as detected through the window 622, detected by the camera 820; thus, the smart catheter 100 is configured such that the discharge fluid within the window housing 620 of the cartridge 620 is only illuminated via the backlight 810 through the window 622. The smart catheter 100 is advantageously further configured to measure turbidity and/or total dissolved solids of the discharge fluid.

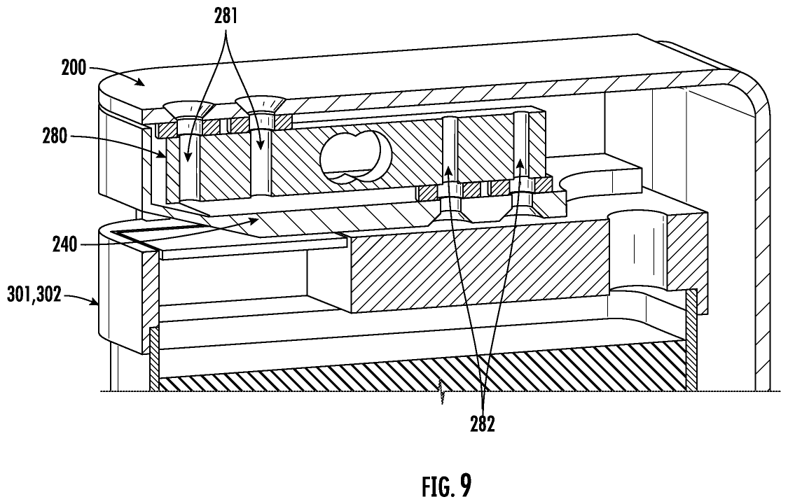

FIGS. 9 and 12 show various aspects of how the tank mount 240 is attached to the frame 200, underneath the region enclosed within the upper shell 220. As shown, a load cell 280 is attached, on a first end thereof (i.e., at the first fastener holes 281), to the frame 200 and, on a second end thereof (i.e., at the second fastener holes 282), to the tank mount 240. Thus, the tank mount 240 is advantageously not directly connected to the frame 200; instead, the tank mount 240 is only connected to the frame 200 via the load cell 280 as an intermediate connecting structure. As the tank 301, 302 that is attached to the tank mount 240 accumulates discharge fluid therein (e.g., from the cartridge 600), the mass and weight of the tank 301, 302 necessarily increases. This increase in mass and weight of the tank 301, 302 correlates directly (e.g., is directly proportional to) the volume of the discharge fluid that is present within the tank 301, 302. As the mass and weight of the tank 301, 302 increases due to accumulation of the discharge fluid within the tank 301, 302, the strain (e.g., deformation) of the load cell 280 increases due to the cantilevered, indirect, attachment of the tank mount 240 to the frame 200 via the load cell 280.

The load cell 280 outputs a signal (e.g., an electrical signal) that corresponds to the strain experienced by the load cell 280 (e.g., exerted thereon by the tank 301, 302 hanging from the tank mount 240 that is connected to the load cell 280). Thus, the strain acting on the load cell 280 directly correlates to the weight of the tank 301, 302. This signal and, thus, the weight and volume of the discharge fluid within the tank 301, 302 can be measured over time to determine the flow rate of the discharge fluid into the smart catheter 100. Thus, the controller 830 is configured to use the signal from the load cell 280 to determine the volume of the discharge fluid within the tank 301, 302 and, as such, to determine both when a tank 301, 302 should be emptied and/or replaced and when to move the valve body 230 of one of the first and second valve assemblies from the open position into the closed position and also to move the valve body 230 of the other of the first and second valve assemblies from the closed position into the open position. Overload protection for the load cell 280 (e.g., a mechanical stop to prevent excess bending deformation of the load cell 280) can be provided in some embodiments, such that damage to the load cell 280 can be prevented.

The cartridge 600 is designed such that the entirety of the cartridge 600 can be removed (e.g., in a unitary manner, in one piece, or without requiring any disassembly of any of the components thereof) from the smart catheter 100, such that a new cartridge 600 can be inserted into the smart catheter 100 prior to use of the smart catheter by a new patient. The ability to entirely remove the cartridge 600 in a single piece is particularly advantageous, since this functionality allows for all components of the smart catheter 100 that are directly contacted by the discharge fluid to be replaced, such that no components of the smart catheter 100 that are in direct contact with the discharge fluid are reused when the smart catheter 100 is deployed for another patient, thus providing a particular hygienic advantage.

FIG. 12 shows further aspects of the smart catheter 100, in which an occupancy sensor, generally designated 250, is provided in a position relative to each of the tank mounts 240 such that, when one of the tanks 301, 302 is fully installed within a corresponding one of the tank mounts 240, a portion of the tank lid 310 of the tank 301, 302 installed on the tank mount 240 is positioned within the detection field of the occupancy sensor 250. In some embodiments, the occupancy detector 250 a break-the-beam type and can be used for detecting any of the position of the door 230, proper engagement of the tank 301, 302 with the tank mount 240, and/or the position of the cartridge 600. Such an occupancy sensor 250 can be particularly advantageously used for detecting the proper installation of one of the tanks 301, 302 in one of the tank mounts 240 because it is possible to perform non-contact presence or occupancy detection, such that the occupancy sensor 250 does not interfere with the accuracy of the weight of the tank 301, 302 by the load cell 280. In such indirect detection embodiments, the occupancy detector 250 can be attached to the frame 200 and/or to the tank mount 240. In embodiments in which the occupancy sensor 250 contacts the tank 301, 302 directly to determine proper installation of the tank 301, 302 with one of the tank mounts 240, the occupancy detector 250 may be attached to the tank mount 240, such that the occupancy sensor 250 floats, along with the tank mount 240, relative to the frame 200, meaning that the occupancy sensor 250 is not attached directly to the frame 200 but is only connected to the frame 200 through the tank mount 240 and the load cell 280.

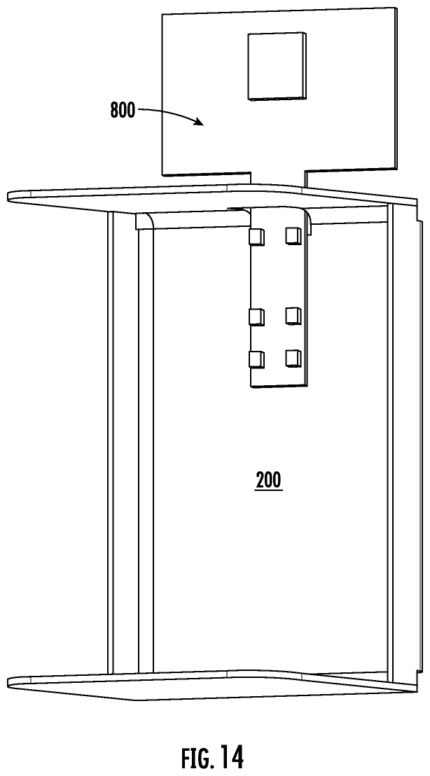

FIGS. 13 and 14 show various aspects of the backplane 800 for the smart catheter 100, as well as for the occupancy sensors 250, the tank level sensors 814, and the tank backlights 812. The controller 830 is configured to control illumination of the backlights 812, individually and/or in unison for one or both tanks 301, 302. The backlights 812 can be controlled so as to be always on, always off, on or off based on the signal received at the controller from the ambient light sensor, or on for a period of time following receiving a user input, such as via the display 150.

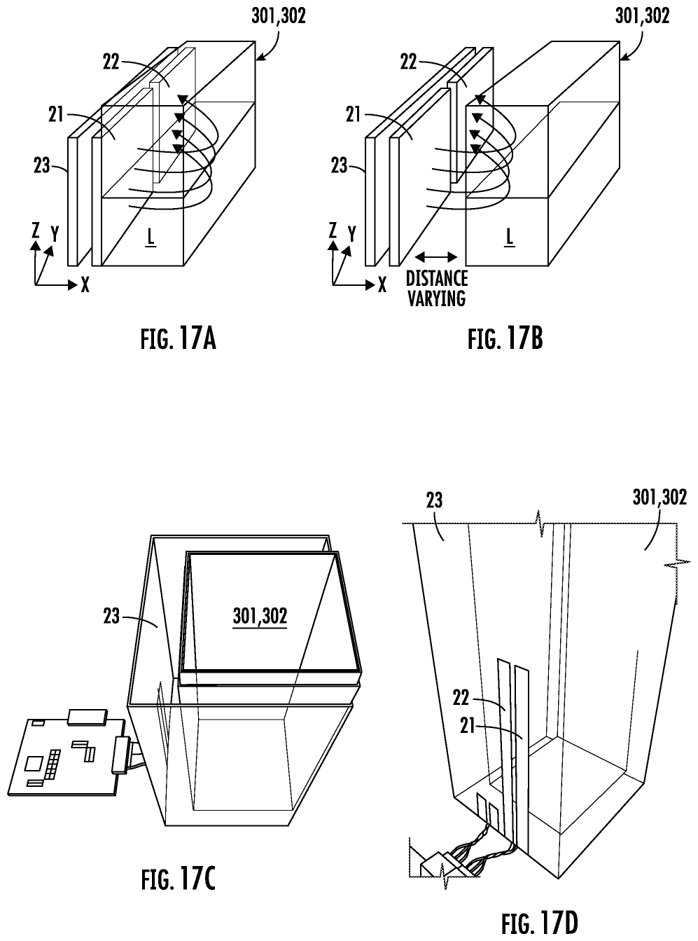

FIGS. 17A-D show various aspects of a system for measuring a level, or volume, of the discharge fluid L within the tank 301, 302. In the example embodiment shown, the system utilizes a capacitive sensor method, such that the volume of discharge fluid L in the tank 301, 302 can be measured either directly or indirectly by such system. FIGS. 17A and 17B are schematic illustrations of systems using direct sensing and remote sensing, respectively. FIGS. 17C and 17D are illustrations of an example embodiment of a remote sensing embodiment. In the direct sensing embodiment shown in FIG. 17A, a sensor electrode 21 and a ground electrode 22 are directly attached to the tank 301, 302 and a shield is provided on an opposite side of the sensor electrode 21 and the ground electrode 22 from the tank 301, 302. A signal is detected, as shown in FIG. 17A, by the signal passing from the sensor electrode 21 to the ground electrode 22 through the discharge fluid L in the tank 301, 302. In the remote sensing embodiment shown in FIG. 17B, a sensor electrode 21 and a ground electrode 22 are spaced apart from the tank 301, 302 by a distance, which can be a variable distance, and a shield is provided on an opposite side of the sensor electrode 21 and the ground electrode 22 from the tank 301, 302. A signal is detected, as shown in FIG. 17B, by the signal passing from the sensor electrode 21 to the ground electrode 22 through the air gap between the sensor and ground electrodes 21,22 and the tank 301, 302 and also through the discharge fluid L in the tank 301, 302. In some embodiments, the controller comprises a DragonBoard 410C, which is a mini PC board. Such a DragonBoard 410C comprises a quad-core processor operating at 1.2 GHz per core, a GPU, 1GB of RAM, 8GB eMMC, WiFi®, Bluetooth®, GPS, Communication (USB, SPI, I2C, UART, etc.), GPIO, and can support a desired operating system (e.g., Android, Linux/Debian, Windows IoT, etc.) The controller can also include a custom adapter PCB, which includes a low speed connector (GPIO—general purpose LED and to supply power) and a high speed connector (MIPI functions); the high speed connector can be used for a USB connection to the camera and a display output for HDMI, SPI0, or MIPI can be provided as well. FIGS. 17C show an example system in which such remote sensing of the level of the discharge fluid L within the tank 301, 302 can be readily determined to allow for the tank 301, 302 to be removed from the smart catheter 100 and replaced with an empty tank 301, 302 prior to the tank 301, 302 overflowing and/or for the controller 830 to trigger actuation of the actuator assemblies 700 to switch into which of the tanks 301, 302 the discharge fluid is dispensed from the cartridge 600.

FIG. 10 shows an example embodiment of the components contained within the upper shell 220 of the smart catheter 100, in which a display 150 (e.g., a touchscreen display) is provided to be both externally visible and provided with touch-based inputs from a user. The camera 820 and the backlight 810 are positioned on opposite sides of the cartridge 600 from each other, such that the backlight 810 emits light that passes through the window 622 of the cartridge 600 to illuminate the discharge fluid contained within the window housing 620, with the camera 820 being oriented to record images of the discharge fluid within the window of the cartridge 600.

In some embodiments, the door 230 comprises one or more transparent slits and/or windows on the front and/or sides of the door 230. These transparent slits and/or windows thus allow for the door 230 to have, at least where such transparent slits and/or windows are provided in the door 230, sufficient local transparency so that both tanks 301, 302 can be viewed from outside the housing of the smart catheter 100 when the door 230 is in its closed position. The door 230 is retained in the closed position in some embodiments by a detent, which can be provided on the frame 200.

The tanks 301, 302 can have graduated markings to indicate a fill level of the discharge fluid within each tank 301, 302. The tanks 301, 302 are advantageously easily removed (e.g., disengaged from the tank mount 240) and replaced (e.g., re-engaged with the tank mount 240). The tanks 301, 302 are advantageously transparent. In the example embodiment, each tank 301, 302 has a 1 liter (L) capacity, with the tank level sensor 814 being configured to detect and trigger an alert and/or switch the cartridge 600 from outputting the discharge fluid from one of the tanks 301, 302 into the other one of the tanks 301, 302, such as when it is detected that the tank is 90% full. The tank level sensor 814 is operably substantially similar to the sensor electrode 21 and the ground electrode 22 shown in FIGS. 17A-D. Thus, the smart catheter 100 has redundancy in measuring the amount of discharge fluid contained within each of the tanks 301, 302, since this can be measured by the load cell 280 and also the tank level sensor 814. The smart catheter 100 can comprise a plurality of vertically-arranged (e.g., spaced apart from each other in the Y-direction) tank level sensors 814, each for determining when a volume of the discharge fluid within the corresponding tank 301, 302 is at one of a plurality of predetermined heights.

Each of the tank mounts 240 comprise a detent feature by which one of the tanks 301, 302 is retained on the tank mount 240 in the proper installed position, thereby preventing undesirable unintended decoupling of the tank 301, 302 from the tank mount 240 during operation of the smart catheter 100. The tanks 301, 302 shown herein are configured to be removed and be emptied manually; however, in some embodiments, the tanks 301, 302 may comprise a drain valve, by which the discharge fluid can be drained out of such tank 301, 302 without such tank 301, 302 needing to be disengaged from the tank mount 240 to which it is attached and/or removed from the smart catheter 100. The operation of the drain valve may be manually controlled or controlled in an automated manner, such as by controller 830. In some embodiments, the tanks 301, 302 can be connected to a respective one of the drain tubes 641, 642 within the cartridge 600 by, for example and without limitation, a dry break quick disconnect.

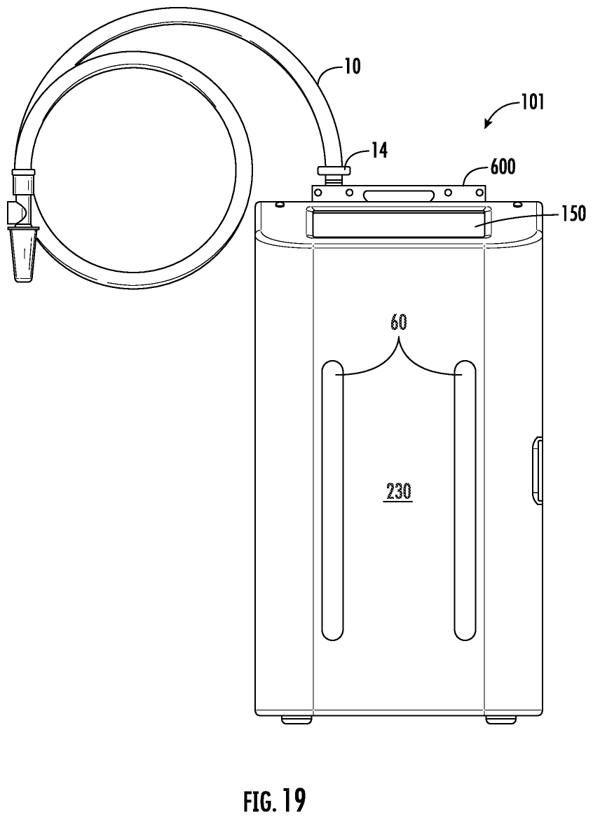

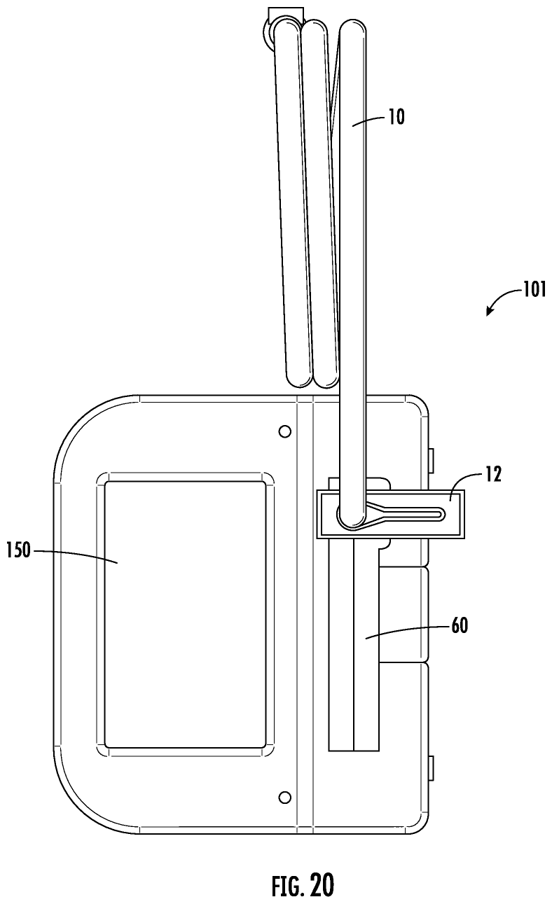

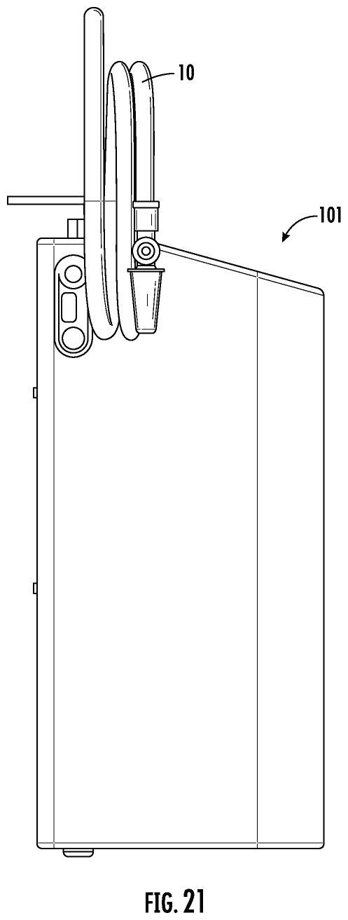

FIGS. 19-21 show various aspects of another example embodiment of a smart catheter, generally designated 101. Unless otherwise described herein as being different, similar structures, features, and/or functions of the smart catheter 101 are substantially identical to those that have already been described herein with respect to the smart catheter 100. As shown in FIG. 19, the smart catheter 101 comprises, protruding out of the top thereof, a cartridge 600. The cartridge 600 comprises a port 14 for attaching the tube 10 to the cartridge 600 for the flow of a discharge fluid into the cartridge 600. Thus, unlike in the smart catheter 100, the smart catheter 101 is designed such that the tube 10 connects to the cartridge 600 in a vertical direction. rather than in the generally horizontal direction shown with respect to the smart catheter 100. The smart catheter 101 is further equipped with a sliding-type clamp 12 through which the tube 10 passes, the clamp 12 being slidable between an open position, in which fluid flow through the tube 10 is unobstructed, and a closed position, in which the side walls of the tube 10 are pressed against each other to prevent fluid flow through the tube 10.

In some embodiments, the presently disclosed smart catheter 100, 101 can be configured as a smart chest tube. Such smart chest tubes can also be referred to herein as precise chest tubes, KG chest tubes, chest tubes, and/or electronic chest tubes. A chest tube is a flexible plastic tube that is inserted through the chest wall and into the pleural space or mediastinum and is used to remove air, fluid, pleural effusion, blood, chyle, or pus from the intrathoracic space. A chest tube is also known as a Bülau drain or an intercostal catheter.

In some cases, pressure around the lungs is lower than atmospheric pressure outside the body. In order to serve as an adequate chest drainage system, the smart catheter 100, 101 is configured to perform the following functions: remove fluid and air promptly, prevent drained air and fluid from returning to the pleural space, and restore negative pressure in the pleural space to re-expand the lung. Thus, in some embodiments, such a smart catheter 100, 101 acting as a smart chest tube is configured to allow air and fluid to leave the chest; comprises a one-way valve to prevent air and fluid from returning to the chest; and comprises a design so that the smart catheter 100, 101 is below the level of the tube 10 (e.g. in the form of a chest tube) for gravity drainage.

An underwater seal chest drainage system can be used to restore proper air pressure to the lungs, re-inflate a collapsed lung as well as remove blood and other fluids. Such an underwater seal chest drainage system is a two-chambered or three-chambered plastic unit with vertical columns bringing measurements marked in milliliters. The thoracic drainage devices cover a wide range and have evolved considerably since their introduction. The basic design principle of such underwater seal chest drainage systems has been the avoidance of air entrance in the pleural cavity during the various phases of the respiratory cycle and continuous drainage of air and fluid from the pleural cavity. The water seal chamber, which is connected in series to the collection chamber, allows air to pass down through a straw or narrow channel and bubble out through the bottom of the water seal. Since air must not return to the patient, a water seal is considered one of the safest and most cost-effective ways for protecting the patient, in addition to being a very useful diagnostic tool. The water seal column can be calibrated to act as a water manometer for measuring intrathoracic pressure.

In a traditional water seal operating system, fluids drain from the patient directly into a large collection chamber via a tube, e.g. a six-foot length of ⅜-inch diameter tube. As drainage fluids collect in this chamber, a nurse or other practitioner can record the amount of fluid that collects on a specified schedule. Disadvantageously, the measuring and recording of the drainage fluid using such systems is, as of yet, done manually by healthcare professionals. Thus, an automated and accurate measurement and recording device is needed to address these known deficiencies.

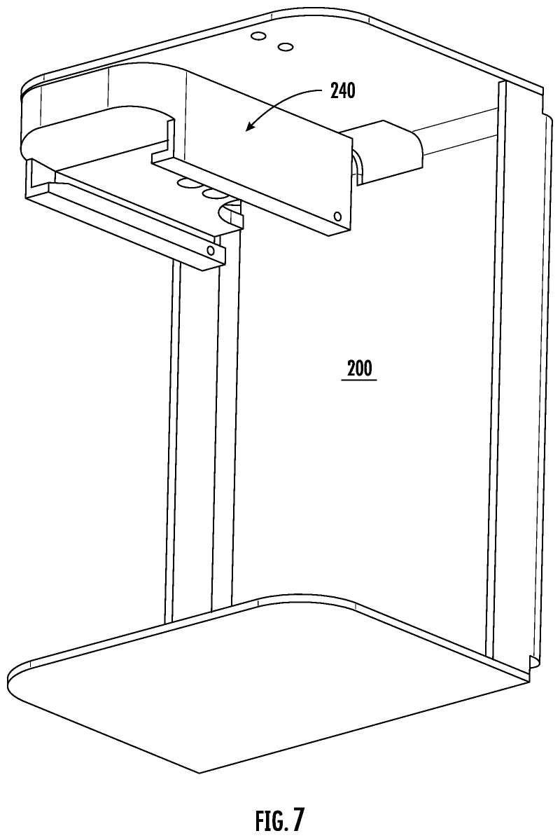

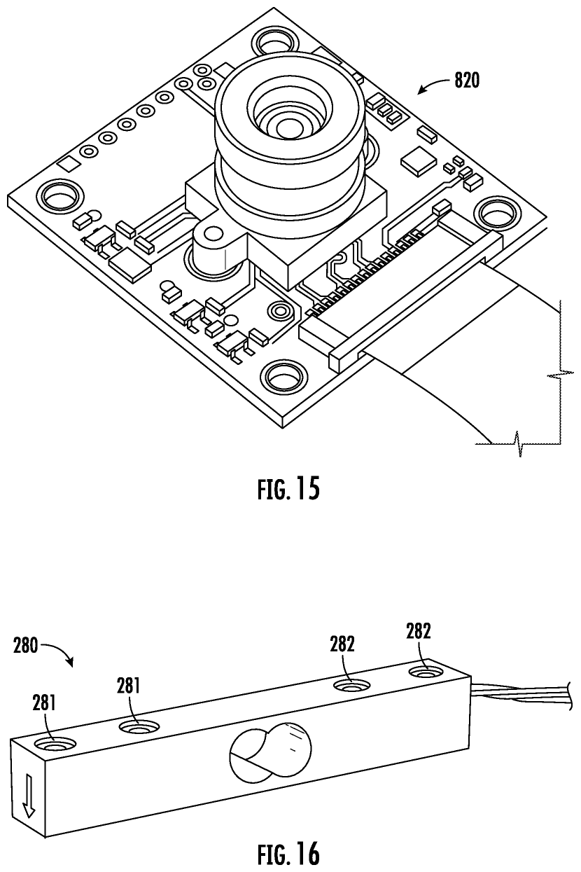

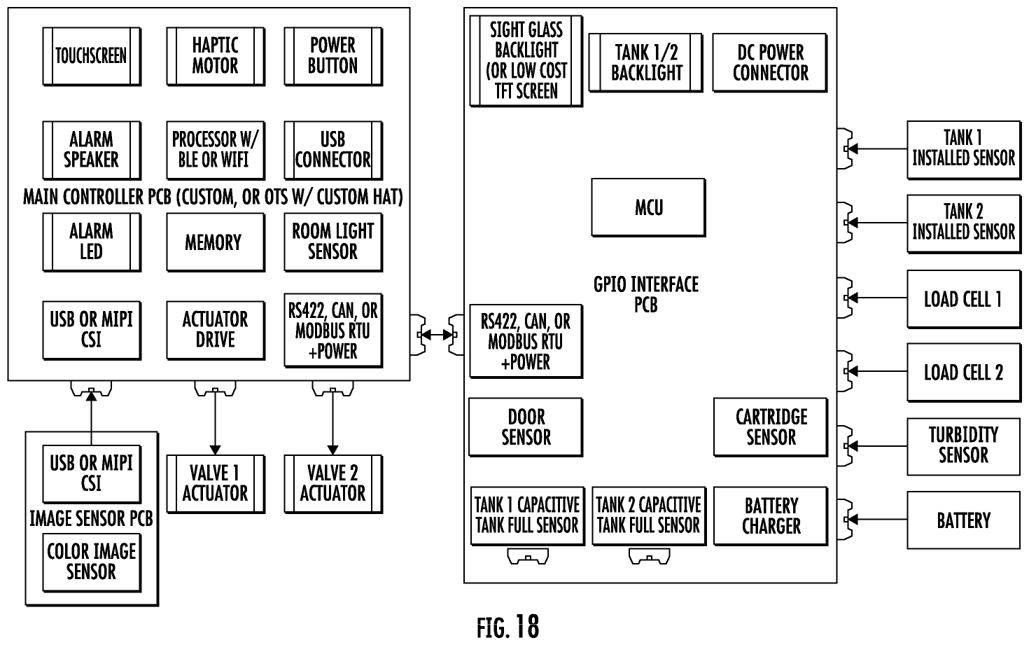

The smart catheter 100, 101 of the present disclosure is operable as a smart chest tube in a manner that is virtually identical to that which is described herein and illustrated in FIGS. 1-21, having essentially all of the same components as the smart catheters 100, 101 of FIGS. 1-21. However, in order for the smart catheters 100, 101 to be configured to act as a smart chest tube, additional components may be required, as those having ordinary skill in the art will fully appreciate, as these components are well known in the art. Specifically, the measuring devices, sensors, and other pieces of equipment described hereinabove can be altered to be able to receive and measure air, fluid, pleural effusion, blood, chyle, or pus flowing from the intrathoracic space through the chest tube. The flexible tube 10 can be replaced or altered to operate as a chest tube, wherein the flexible tube 10 comprises one or more openings at one end, and a drainage port at the opposing end. In some embodiments, the one or more openings can be located at the end face of the flexible tube, or they can be axially aligned, meaning they run down the length of the flexible tube 10, starting near the end. In some other embodiments, the one or more openings can be circumferentially aligned, meaning around the circumference of the flexible tube 10.

In some embodiments, the smart catheter 100, 101 is configured to measure an amount of air, fluid, pleural effusion, blood, chyle, and/or pus flowing therethrough. The smart catheter 100, 101 can be adapted to properly measure almost any fluid flowing therethrough and is not necessarily restricted to the measurement of only liquid(s). The measured volume of air, fluid, pleural effusion, blood, chyle, and/or pus determined by the smart catheter 100, 101 can be transmitted as data to an external device (e.g. a computer or receiver). As described herein, the smart catheter 100, 101 can, when configured as a smart chest tube device, in some embodiments comprise a transmitter or transmission device to wirelessly transmit the data to an external device. The smart catheter 100, 101 can, when configured as a smart chest tube device, in some embodiments also comprise a receiver configured to receive data from an external device.

In some embodiments, when the smart catheter 100, 101 is operable as a smart chest tube, the smart catheter 100, 101 is configured to measure and calculate the volume of air, fluid, pleural effusion, blood, chyle, and/or pus produced by a patient and activate an alarm if such volume output exceeds or does not meet a set or predetermined threshold or, as described above, if one or both of the tanks 301,302 are full, about to be full, or if there is a malfunction. All of the features and aspects described elsewhere herein with respect to the smart catheter 100, 101 can be adapted and/or altered to account for any differences in the operations of a catheter (e.g., a urinary catheter) versus a chest tube. For example, tubes and ports sizes can be increased and materials can be altered to make it more available for pus and chyle storage.

Additionally, for the smart chest tube application, the smart catheter 100, 101 as described herein can comprise a suction control chamber to aid in applying suction (e.g., applying a vacuum, or negative pressure to) the fluid being drained from the chest cavity of the patient.

In some embodiments, the smart catheter 100, 101 is configured to detect and calculate a flow rate of the discharge fluid flowing into and/or out of (e.g., through) the smart catheter 100, 101 (e.g., into the cartridge 600 and, subsequently, into one of the tanks). In an advantageous embodiment, the flow rate of the discharge fluid is calculated as a trailing average with a preferable resolution of 5 mL. The time increment of measurement and also the total time period of measurement can be specified (e.g., 15 minute increments over an 8 hour time period).

In some embodiments, the smart catheter 100, 101 can comprise one or more rubber feet on the bottom (e.g., on the lowest surface of the base 210) thereof such that, if the smart catheter 100, 101 were positioned on a surface, any sliding of the smart catheter 100, 101 relative to this surface would be minimized. In some embodiments, the smart catheter 100, 101 comprises a clamp and/or bracket on the back surface thereof (e.g., on the opposite side of the frame 200 from the tank region 300), the clamp and/or bracket being configured to be rotated such that the opening of such clamp and/or bracket is either parallel to the length of the housing of the smart catheter 100, 101 or perpendicular to the length of the housing of the smart catheter 100, 101. Those having ordinary skill in the art will appreciate that the clamp and/or bracket can be adjusted up or down along a track on the back of the smart catheter 100, 101 as well. Moreover, in some embodiments, the clamp and/or bracket is rotatable in place and does not need to be removed in order to rotate it to be parallel with or perpendicular with respect to the smart catheter. Furthermore, in some embodiments, the clamp and/or bracket can be configured to mount the smart catheter 100, 101 to a standard hospital bed frame, transport poles, or any other suitable location. For example and without limitation, the clamp or bracket can be configured to attach the housing of the smart catheter 100, 101 or enclosure to a bed or other frame, pole, or other suitable attachment point with dimensions of, for example and without limitation, ½″, 1″, 1½″, 2″, 2½″, etc.

In some embodiments, the smart catheter 100, 101 of disclosed herein is configured to present various pieces of information on the display 150. For example, and without limitation, the display 150 can be configured to show information identifying the patient for which the smart catheter 100, 101 is being used for. Such identifying information could be a picture or avatar of the patient or the patient's name, or patient identification number. In some embodiments, operational data of the smart catheter 100, 101, including, for example and without limitation, patient name, vital, and/or demographic information, discharge fluid flow rate, alarm events, etc. can be logged in a data storage module of the smart catheter 100, 101. This logged operational data can be accessible via a wireless and/or wired communication protocol, including universal serial bus (USB). In some embodiments, the smart catheter 100, 101 is configured to store in a data storage device thereof one or more of (e.g., all of) patient name, patient medical record number, patient date of birth, patient gender, patient dialysis status.

In some embodiments, fluid levels or capacities of each of the tanks 301, 302, or collection reservoir, can be displayed. In some embodiments, the one or more processors situated within the housing of the smart catheter 100, 101 can control what and when items are displayed on the display 150. In some embodiments, the alarms described herein can coincide with various indicators flashing on the display to help alert healthcare workers of the issue. Additionally, in some embodiments, the display 150 can display (e.g., temporarily or for a prescribed period of time, or until reset) the fluid level indicator of one more of the tanks 301, 302 when the volume of the discharge fluid contained therein has reached one or more designated thresholds. In this way, medical staff can easily determine which of the tanks 301, 302 is full before even opening the door 230. In some embodiments, the display 150 can also show pertinent information about the discharge fluid, such as, for example, any dissolved solids statistics or other information pertaining thereto. In some embodiments, the display 150 is configured to illuminate or otherwise to show a color that corresponds to (e.g., is the same as or substantially similar to) the color of the discharge fluid. In some further embodiments, the display 150 has the ability to have sensor motion to turn the light 60 on at any desired light intensity, ranging from dim to bright light.

Those having ordinary skill in the art will appreciate that the display 150 can be configured to display any appropriate information that relates to the patient, medical workers, the status of the smart catheter 100, 101 or any of its parts, etc. To allow a medical professional who is operating and monitoring the smart catheter 100, 101 to have easier control over the smart catheter 100, 101, the display 150 can be a touchscreen display that has multiple different pages, folders, and buttons that can be displayed, changed, altered, customized, etc. Additionally, in some embodiments, the one or more processors can be in communication with one or more external devices. In some embodiments, the external devices comprise servers or other computers hosting the patient's medical records and/or medical chart. Such external devices can comprise a tablet, computer, mobile device, phone, smart watch, audio device, handheld documentation device, and/or display device. In some such embodiments, the one or more processors can be configured to automatically measure the amount or volume of the discharge fluid (e.g., urine) according to predetermined sets of time (e.g., continuously, periodically, randomly, every 30 minutes or one hour, etc.) in one or more of the tanks 301, 302 and to transmit a volume of the discharge fluid in one or both of the tanks 301, 302 and/or a total volume that has been discharged over a period of time, which can be predefined or user-defined. Additionally, information about the discharge fluid determined from the various sensors can also be recorded and sent to the medical records and/or medical chart for updating.

In some embodiments such a smart catheter 100, 101 as provided herein further comprises a power source, a computer, memory, a receiver or transmitter, an accelerometer, a speaker, microphone, or a tactile signal device, wherein the power source, computer, memory, receiver or transmitter, accelerometer, speaker or tactile signal device are interconnected with one another. In some embodiments, such a smart catheter 100, 101 as provided herein further comprises a computer program product comprising computer executable instructions embodied in a computer readable medium for performing steps comprising receiving an electrical signal from a measuring apparatus, processing the electrical signal to calculate data pertaining to a measured volume, and relaying the data to the electronic display, speaker, tactile signal device, and/or external device. In some embodiments, a wireless receiver is configured to receive data wirelessly and transfer it to a computer, wherein the computer is configured to process the data and transmit it to the display, speaker, and/or tactile signal device.

The functions and subject matter described herein, especially with respect to the one or more processors described herein, can in some embodiments be implemented using a computer program product comprising computer executable instructions embodied in a computer readable medium. Such computer readable medium can be stored in memory and implemented by computer. Exemplary computer readable media suitable for implementing the subject matter described herein include disk memory devices, chip memory devices, application specific integrated circuits, programmable logic devices, and downloadable electrical signals. In addition, a computer program product that implements the subject matter described herein may be located on a single device or computing platform or may be distributed across multiple devices or computing platforms.

All of the measurements and/or transmissions disclosed herein can be performed in real time (e.g., as soon as the discharge fluid flows and stops flowing into the tank, the smart catheter can transmit the volume data and discharge fluid characteristics data to the medical records server), at a prescribed schedule, and/or upon receiving a command from a user of the smart catheter 100, 101. In some embodiments, the smart catheter 100, 101 can comprise a transmitter, transmission device, and/or transceiver for making a wired or wireless connection to the medical records and/or medical chart server/computer. Moreover, in some embodiments, the smart catheter 100, 101 comprises a receiver, such as, for example and without limitation, a wireless and/or wired receiver configured to receive data from any of the external devices disclosed herein. Using the one or more processors and the transmitter and/or the receiver, any component of the smart catheter 100, 101 can receive or transmit data to/from any suitable external or internal device (e.g., the medical records or medical chart server, mobile phones, tablets, medical equipment, etc.). A wireless receiver and/or transmitter can be configured to wirelessly receive and/or transmit data and information via wireless signal. By way of example and not limitation, such wireless forms of communication can comprise WiFi® and Bluetooth®. As described herein, with integrated wireless communication capabilities, the smart catheter 100, 101 disclosed herein can exchange information and/or data (e.g., to receive and/or transmit) with another device, such as, but not limited to a tablet, computer, phone, smart watch, audio device, or display device.

In some embodiments, the smart catheter 100, 101 can be powered by a wired power cable connected to an electrical outlet. In further embodiments, the smart catheter 100, 101 can be powered by a battery or other suitable power source. In either embodiment, the smart catheter 100, 101 can be configured to trigger and sound an alarm as described herein when either the power connection is inadequate, or there is a malfunction with the power connector or the battery or other power source. Additionally, in some embodiments, the smart catheter 100, 101 can be operable using speech recognition. In support of this feature, some embodiments of the smart catheter 100, 101 of the present disclosure comprise speakers and an audio input device, such as a microphone, that allow a user to speak to the smart catheter to issue commands or requests. Moreover, in such an embodiment, the one or more processors of the smart catheter 100, 101 can be configured to operate an artificial intelligence program that is configured to receive spoken commands and respond with additional audio feedback or perform tasks in connection with the commands. In this way, any of the parameters, actions, services, or performances that can be performed automatically, or manually be a person touching the smart catheter 100, 101 can also be performed via voice command using speech recognition.

In some embodiments, the smart catheter 100, 101 is configured to display a graphical user interface (GUI) on the display 150 thereof, the GUI being designed to prompt a user (e.g., a healthcare professional) through the steps that are required to be performed when the smart catheter 100, 101 is being assigned and deployed for use with a new patient. When selecting initialization of the smart catheter 100, 101, the user is guided through steps that can include patient information data entry, initialization steps for installing new tanks 301, 302, and/or installation of a new cartridge 600.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

1. A smart fluid discharge monitoring device comprising:
a housing;
a frame within the housing;
a cartridge positioned within an upper shell of the housing, wherein the cartridge comprises a window, through which a discharge fluid is configured to flow;
a door removably attached to the housing, the door delimiting an interior region of the housing below the upper shell;
a tube connected to the cartridge; and
a plurality of tanks removably secured within the interior region, wherein the tanks are configured to receive the discharge fluid from the cartridge;
wherein the smart fluid discharge monitoring device is configured to detect one or more optical characteristics of the discharge fluid through the window as the discharge fluid flows through the cartridge.

2. The smart fluid discharge monitoring device of claim 1, comprising:
a controller;
wherein the cartridge comprises a valve assembly for each of the plurality of tanks; and
wherein the controller is configured to control actuation of the valve assemblies between an open position and a closed position.

3. The smart fluid discharge monitoring device of claim 2, comprising a tank occupancy sensor configured to detect when a tank is installed within the smart fluid discharge monitoring device.

4. The smart fluid discharge monitoring device of claim 3, wherein:
each tank is attached to the frame via a corresponding tank mount;
each tank mount is connected to the frame by a load cell; and
the load cell is configured to output a signal that corresponds to a volume of the discharge fluid within the tank attached to the corresponding tank mount

5. The smart fluid discharge monitoring device of claim 4, wherein the controller receives the signal from the load cell and, when the controller determines that one of the tanks is full, or within a predefined percentage of full, the controller is configured to close the valve assembly for the full tank and to open the valve assembly of another of the tanks.

6. The smart fluid discharge monitoring device of claim 4, wherein each tank mount is connected to the frame only by the load cell, in a cantilevered manner.

7. The smart fluid discharge monitoring device of claim 2, wherein each valve assembly of the cartridge comprises an actuator configured to engage with and move a valve body of a same valve assembly between an open position, in which the valve body is positioned to allow the discharge fluid to flow into a corresponding one of the plurality of tanks from the cartridge, and a closed position, in which the valve body is positioned to block the discharge fluid from flowing into the corresponding one of the plurality of tanks from the cartridge.

8. The smart fluid discharge monitoring device of claim 7, wherein, for each valve assembly of the cartridge, the valve body comprises a sloped surface against which the actuator is configured to engage in a sliding or rolling manner as the valve body moves between the open and closed positions.

9. The smart fluid discharge monitoring device of claim 8, wherein:
each valve assembly comprises an arm that is attached to the actuator;
for each valve assembly of the cartridge, the valve body comprises, at an end of the sloped surface that corresponds to the open position for the valve body when engaged by the actuator, a recess;
the arm comprises, at a distal end thereof, a roller; and
when the roller is engaged within the recess, the valve body is held in the open position until the actuator is energized to retract the arm, which disengages the roller from the recess.

10. The smart fluid discharge monitoring device of claim 9, wherein each valve assembly is configured to default to the valve body being in the closed position unless the roller is engaged within the recess.

11. The smart fluid discharge monitoring device of claim 1, wherein the smart fluid discharge monitoring device is configured to measure a flow rate of the discharge fluid through the cartridge.

12. The smart fluid discharge monitoring device of claim 1, comprising a backlight configured to illuminate the discharge fluid through the window and a camera configured to detect the one or more optical characteristics of the discharge fluid through the window.

13. The smart fluid discharge monitoring device of claim 12, wherein the one or more optical characteristics comprise turbidity and/or total dissolved solids.

14. The smart fluid discharge monitoring device of claim 12, wherein the cartridge is positioned within the upper shell, which is configured to block exterior light from entering into the upper shell.

15. The smart fluid discharge monitoring device of claim 1, wherein the smart fluid discharge monitoring device is a smart catheter, configured for connection to a catheter configured for evacuation of the discharge fluid from a patient to whom the catheter is attached.

16. The smart fluid discharge monitoring device claim 1, wherein the smart fluid discharge monitoring device is a smart chest tube, configured for connection to a chest tube configured for evacuation of the discharge fluid from a patient to whom the chest tube is attached.

17. A method of monitoring a discharge fluid from a patient, the method comprising:
providing a smart fluid discharge monitoring device, comprising:
a housing;
a frame within the housing;
a cartridge positioned within an upper shell of the housing, wherein the cartridge comprises a window, through which a discharge fluid is configured to flow;
a door removably attached to the housing, the door delimiting an interior region of the housing below the upper shell;
a tube connected to the cartridge; and
a plurality of tanks removably secured within the interior region;
connecting a discharge tube attached to a patient to the tube of the cartridge;
detecting one or more optical characteristics of the discharge fluid through the window as the discharge fluid flows through the cartridge; and
receiving the discharge fluid into one of the tanks from the cartridge.

18. The method of claim 17, wherein the cartridge comprises a valve assembly for each of the plurality of tanks, the method comprising providing a controller that controls actuation of the valve assemblies between an open position and a closed position.

19. The method of claim 18, comprising detecting, via a tank occupancy sensor, when a tank is installed within the smart fluid discharge monitoring device.

20. The method of claim 19, comprising:
attaching each tank to the frame via a corresponding tank mount;
connecting each tank mount to the frame by a load cell; and
outputting, from the load cell, a signal that corresponds to a volume of the discharge fluid within the tank attached to the corresponding tank mount

21. The method of claim 20, comprising receiving, at the controller, the signal from the load cell and, when the controller determines that one of the tanks is full, or within a predefined percentage of full, closing, using the controller, the valve assembly for the full tank and opening, using the controller, the valve assembly of another of the tanks.

22. The method of claim 20, wherein each tank mount is connected to the frame only by the load cell, in a cantilevered manner.

23. The method of claim 18, wherein each valve assembly of the cartridge comprises an actuator that engages with a valve body of a same valve assembly, the method comprising using the actuator to move the valve body between an open position, in which the valve body is positioned to allow the discharge fluid to flow into a corresponding one of the plurality of tanks from the cartridge, and a closed position, in which the valve body is positioned to block the discharge fluid from flowing into the corresponding one of the plurality of tanks from the cartridge.

24. The method of claim 23, wherein, for each valve assembly of the cartridge, the valve body comprises a sloped surface against which the actuator engages in a sliding or rolling manner as the valve body moves between the open and closed positions.

25. The method of claim 24, wherein:
each valve assembly comprises an arm that is attached to the actuator;
for each valve assembly of the cartridge, the valve body comprises, at an end of the sloped surface that corresponds to the open position for the valve body when engaged by the actuator, a recess;
the arm comprises, at a distal end thereof, a roller; and
when the roller is engaged within the recess, the valve body is held in the open position until the actuator is energized to retract the arm, which disengages the roller from the recess.

26. The method of claim 25, wherein each valve assembly defaults to the valve body being in the closed position unless the roller is engaged within the recess.

27. The method of claim 17, comprising measuring a flow rate of the discharge fluid through the cartridge.

28. The method of claim 17, comprising:
illuminating, using a backlight, the discharge fluid through the window; and
detecting, using a camera, the one or more optical characteristics of the discharge fluid through the window.

29. The method of claim 28, wherein the one or more optical characteristics comprise turbidity and/or total dissolved solids.

30. The method of claim 28, wherein the cartridge is positioned within the upper shell, which blocks exterior light from entering into the upper shell.

31. The method of any of claim 17, wherein the discharge tube is a catheter and the smart fluid discharge monitoring device is a smart catheter.

32. The method of claim 17, wherein the discharge tube is a chest tube and the smart fluid discharge monitoring device is a smart chest tube.
